(19) United States
(12) Patent Application Publication
(10) Pub. No.: US 2018/0143145 A1
(43) Pub. Date: May 24, 2018

(54) SENSOR FOR PERFORMING DIELECTRIC MEASUREMENTS

(71) Applicant: Imperial Innovations Limited, London (GB)

(72) Inventors: Norbert Klein, Staines-upon-Thames (GB); Clare Watts, West Yorkshire (GB); Stephen Hanham, London (GB)

(73) Assignee: Imperial Innovations Limited, London (GB)

(21) Appl. No.: 15/359,876

(22) Filed: Nov. 23, 2016

(51) Int. Cl. G01N 22/00; G01N 27/06; G01N 33/18

(52) U.S. Cl. CPC G01N 22/00; G01N 33/18; G01N 27/06

(57) ABSTRACT

A sensor comprises a first resonator operable at microwave and/or millimetre wave frequencies and having a first value of quality factor which is equal to or greater than moo at a frequency in a range 1 to 100 GHz and a second resonator having a second value of quality factor which is less than the first value of quality factor and which is positioned and orientated with respect to the first resonator so as to be inductively coupled to the first resonator, the second resonator comprising first and second electrically-conductive regions separated by a gap which provides a sensing region.

### FIELD OF THE INVENTION

The present invention relates to a sensor for performing dielectric measurements at microwave or millimetre wave frequencies.

### BACKGROUND

Sensors operating at microwave frequencies can be highly sensitive when measuring the complex dielectric permittivity, ε*, of small volumes of aqueous solutions due to the strong interaction of water with electric fields at these frequencies. Thus, they show promise for biosensing applications where most liquids to be investigated consist predominantly of water.

Coaxial probe approaches have shown to be sensitive to low concentrations of biomolecules over a broad frequency range, and concentration-independent differentiation between different dissolved protein species was demonstrated based on small variations in the real and imaginary components of complex dielectric permittivity ε* around the Debye relaxation frequency of water and reference is made to T. H. Basey-Fisher et al.: “Microwave Debye relaxation analysis of dissolved proteins: Towards free-solution biosensing”, Applied Physics Letters, volume 99, page 233703 (2011).

Microwave dielectric resonators operating in whispering gallery modes at 10-40 GHz have been used to distinguish low concentrations of organic liquids and solutions of proteins, glucose (<0.1% w/w) and sodium chloride (70 ppt) using sub-nanoliter volumes of analyte and reference is made to E. N. Shaforost et al.: “Nanoliter liquid characterization by open whispering-gallery mode dielectric resonators at millimeter wave frequencies”, Journal of Applied Physics, volume 104, page 074111 (2008) and E. N. Shaforost et al.: “High sensitivity microwave characterization of organic molecule solutions of nanoliter volume,” Applied Physics Letters, volume 94, page 112901 (2009).

Other approaches using on-chip electrodes and microfluidic channels to concentrate electric fields around the liquid under test have achieved high sensitivity to ethanol solutions on volumes less than 0.5 nL, as well as showing promise for cell sensing. Reference is made to T. Chretiennot et al.: “A microwave and microfluidic planar resonator for efficient and accurate complex permittivity characterization of aqueous solutions,” IEEE Transactions on Microwave Theory and Techniques, volume 61, pages 972-978 (2013) and K. Grenier et al.: “Integrated Broadband Microwave and Microfluidic Sensor Dedicated to Bioengineering,” IEEE Transactions on Microwave Theory and Techniques, volume 61, pages 3246-3253 (2009).

Resonant split-ring structures have also been used to demonstrate significant sensitivity for aqueous solutions of organic liquids and biomolecules. Reference is made to W. Withayachumnankul et al.: “Metamaterial-based microfluidic sensor for dielectric characterization,” Sensors Actuators, A Phys., volume 189, pages 233-237 (2013), A. Abduljabar et al.: “Novel microwave microfluidic sensor using a microstrip split-ring resonator,” IEEE Transactions on Microwave Theory and Techniques, volume 62, pages 679-688 (2014) and H. Torun et al.: “An antenna-coupled split-ring resonator for biosensing”, Journal of Applied Physics, volume 116, page 124701 (2014).

Ali A. Abduljabar et al.: “Modelling and Measurements of the Microwave Dielectric Properties of Microspheres”, IEEE Transactions on Microwave Theory and Techniques, volume 63, no. 12 (2015) describes employing a split ring resonator to measure the dielectric constant of a single dielectric particle inside a water-filled quartz capillary. This system, however, requires careful mechanical alignment of capillaries and so is not particularly compatible with a lab-on-a-chip. Moreover, the low-quality factor of the split-ring requires broadband frequency sweeps to measure subtle cell-induced changes of resonant parameters. This increases data acquisition time and, thus, limits throughput.

A. Ferrier et al.: “A microwave interferometric system for simultaneous actuation and detection of single biological cells,” Lab on a Chip, volume 9, pages 3406-3412 (2009) describes an electrical approach for single-cell analysis based on detection of cell-induced capacitance changes. This approach, however, requires microwave-suitable electrical contacts to be made with the integrated microfluidic circuit. Moreover, the system uses a microwave frequency at which the cell membrane may still screen the cell interior by the membrane.

In Y. Yang et al.: “Distinguishing the viability of a single yeast cell with an ultra-sensitive radio frequency sensor,” Lab on a Chip, volume 10, pages 553-555 (2010), an interferometer approach based on a coplanar waveguide was demonstrated for a measurement frequency of 5 GHz. Similar to A. Ferrier et al. ibid., electrical contacts are required for microwave coupling (in that particular case, by a coplanar wafer probe) which requires manual adjustment under a microscope. Therefore, this technique is not particularly suited for realizing a low-cost lab-on-chip.

Standard broadband coplanar techniques, such as those described in D. Dubucet et al.: “Microwave-based biosensor for on-chip biological cell analysis,” Analog Integrated Circuits and Signal Processing, volume 77, pages 135-142, (2013) and Y. Ning et al.: “Broadband electrical detection of individual biological cells,” IEEE Transactions on Microwave Theory and Techniques, volume 62, pages 1905-1911 (2014), suffer from insufficient sensitivity for single cell detection and also require slow broadband measurements and advanced electrical contacts.

### SUMMARY

According to a first aspect of the present invention there is provided a sensor comprising a first resonator operable at microwave or millimetre wave frequencies and having a first value of quality factor which is equal to or greater than woo at a frequency in a range between 1 and 100 GHz and a second resonator having a second value of quality factor which is less than the first value of quality factor and which is positioned and orientated with respect to the first resonator so as to be inductively coupled to the first resonator. The second resonator comprises first and second electrically-conductive regions separated by a gap which provides a sensing region.

The quality factor of the first resonator may be equal to or greater than 2000 or may be equal to or greater than 5000.

The quality factor of the second resonator may be greater than or equal to 1 and/or equal to or less than 500.

The first resonator may comprise a dielectric resonator. The dielectric resonator may comprise a material having a dielectric constant greater than or equal to 3, greater than or equal to 10 or greater than or equal to 20. The dielectric resonator may have a temperature coefficient of the resonant frequency less than or equal to 10 ppm/° C. between 25 and 60° C. The dielectric resonator may have a temperature coefficient of the resonant frequency greater than 10 ppm/° C. between 25 and 60° C. The dielectric resonator material may be barium zinc tantalate. The first resonator may be generally cylindrical. The first resonator may comprise a conductive housing defining a cavity and an aperture in the housing disposed between the cavity and the second resonator. The cavity is generally cylindrical and the aperture is circular.

The second resonator may be generally planar. The second resonator may comprise a ring comprising at least gap in the ring and is able to support rotational modes around the ring. The second resonator may be a split-ring resonator. The second resonator may comprise co-planar thin film regions of conductive material. The sensing region includes an out-of-plane region.

According to a second aspect of the present invention there is provided a sensor system comprising the sensor and a measurement circuit coupled to the first resonator so as to excite a resonant mode in the first resonator.

The measurement system may comprise a vector network analyser, such as a Hewlett Packard 8752 A or a loop oscillator of which the first resonator represents the frequency stabilizing element.

The sensor system may further comprise at least one electrical probe arranged to couple energy into the first and second resonators. The sensor system may further comprise at least one magnetic probe arranged to couple energy into the first and second resonators. The sensor system may further comprise at least waveguide arranged to couple energy into the first and second resonators.

The second resonator may comprise a ring comprising at least gap in the ring and is able to support rotational modes around the ring and wherein the measurement circuit is arranged to excite a rotational mode in the second resonator.

The sensor system may be configured to excite a resonance mode at a frequency which is equal to or greater than 5 GHz, is equal to or greater than 10 GHz, which is equal to or greater than 40 GHz, which is equal to or greater than 100 GHz.

The sensor system may be configured to excite a resonance mode at a frequency which is equal to or less than 100 GHz, which is equal to or less than 200 GHz, which is equal to or less than 500 GHz or equal to or less than 1 THz.

The measurement system may be arranged to determine a resonant frequency and a quality factor of a coupled mode using transmission or reflection measurements based on S-parameter analysis of the cavity.

The sensor system may further comprise a microfluidic chip comprising at least one microfluidic channel, wherein the chip is arranged such that the channel is disposed sufficiently close to sensing region so as to influence a resonant mode in the second resonator.

The channel may have a side which is exposed directly to the second resonator.

The sensing system may be arranged so as to be a table-top system. The sensing system may be a portable system. The system may be hand-portable or hand-holdable system.

According to a third aspect of the present invention there is provided a flow cytometer comprising the sensor system.

According to a fourth aspect of the present invention there is provided a biological sensing system comprising the sensor system.

According to a fifth aspect of the present invention there is provided an arrangement of a dielectric resonator inside a metallic shielding cavity and a microfluidic chip on top of the cavity such that the microfluidic chip contains a microfluidic channel that passes through at least one capacitive gap of a metallic split ring resonator.

At least one electromagnetic resonant modes of each of the dielectric resonator and the split ring resonator may be mutually coupled via an aperture inside the cavity. A circular aperture may be aligned with a cylindrical dielectric resonator and a circular split ring. The dielectric resonator may be excited in a mode of rotational symmetry (TE_{o,n,p+δ} mode where n and p are positive integers) and the split ring resonator may have two oppositely faced gaps with a straight microfluidic channel passing through both gaps. The split ring resonator may be realized as patterned thin film made from a metal of high conductivity. The coupled dielectric resonator—split ring resonator mode may be excited and detected by at least one coaxial electric or magnetic probe or by a waveguide aperture inside the cavity for operation frequencies up to about 100 GHz.

The resonant frequency and quality factor of coupled modes may be determined by transmission or reflection measurements based on S-parameter analysis of the cavity only. The dielectric resonator may be machined from a microwave ceramic material comprising high dielectric constant and low temperature coefficient of the resonant frequency. The microfluidic chip may be composed of an open channel prepared by moulding, photoresist patterning or deep etching and a cover slip which contains the metallic split ring resonator prepared by thin film deposition and patterning.

### BRIEF DESCRIPTION OF THE DRAWINGS

Certain embodiments of the present invention will now be described, by way of example, with reference to the accompanying drawings, in which:

FIG. 1 is an exploded perspective view of a sensor system comprising a sensor, a microfluidic chip and measurement system;

FIG. 2 is a perspective view of the sensor and microfluidic chip shown in FIG. 1;

FIG. 3 is cross-sectional view of a part of a multi-piece conductive housing which defines a cavity and a dielectric resonator disposed within the cavity;

FIG. 4 is a plan view of a split-ring resonator;

FIG. 5 is a cross-sectional view of the split-ring resonator shown in FIG. 4 taken along the line A-A′;

FIG. 6 illustrates simulated magnetic field distribution of a coupled mode illustrating inductive coupling between a dielectric resonator and a split-ring resonator;

FIG. 7 illustrates simulated electric field distribution of a coupled mode illustrating electric is field enhancement within gaps in a split-ring resonator;

FIG. 8 is a plot of measured quality factor against time showing detection of a polystyrene particle in water passing through a microfluidic channel;

FIG. 9 is an equivalent circuit for a sensor system comprising a dielectric resonator and a split-ring resonator; and

FIG. 10 illustrates changes in frequency response due to the presence of target, such as a cell.

### DETAILED DESCRIPTION OF CERTAIN EMBODIMENTS

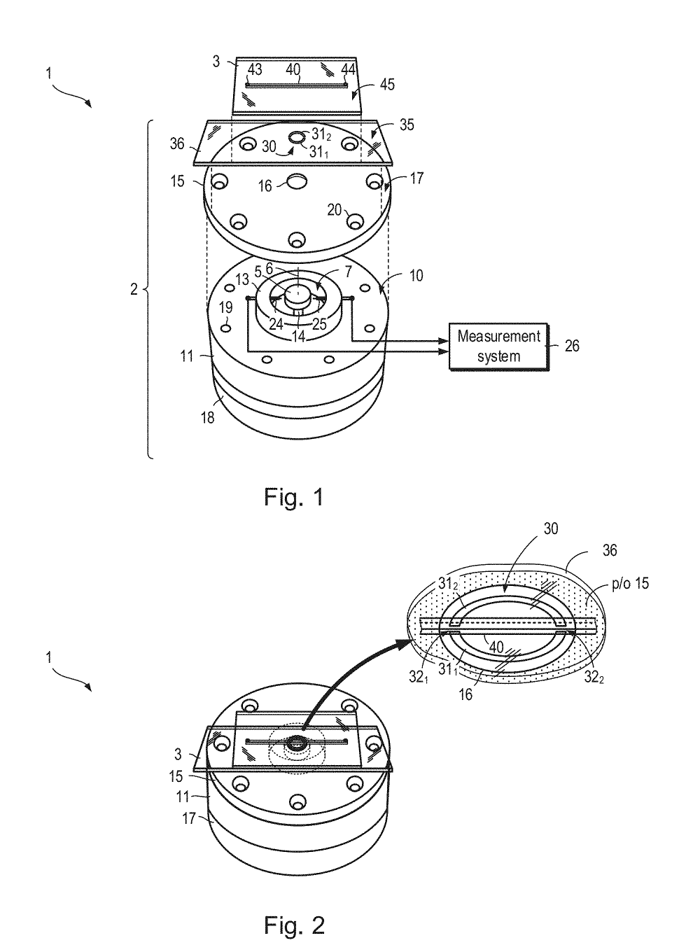

Referring to FIGS. 1 and 2, a sensor system 1 for performing dielectric measurements at microwave and/or millimetre wave frequencies is shown. The sensor system 1 can be used to sense, detect or measure targets, such as a specific type of cell (for example a cancerous cell) or other specific types of biochemical or chemical materials or particles.

The sensor system 1 includes a coupled resonator arrangement 2 (herein referred to simply as “the sensor”) and a microfluidic chip 3.

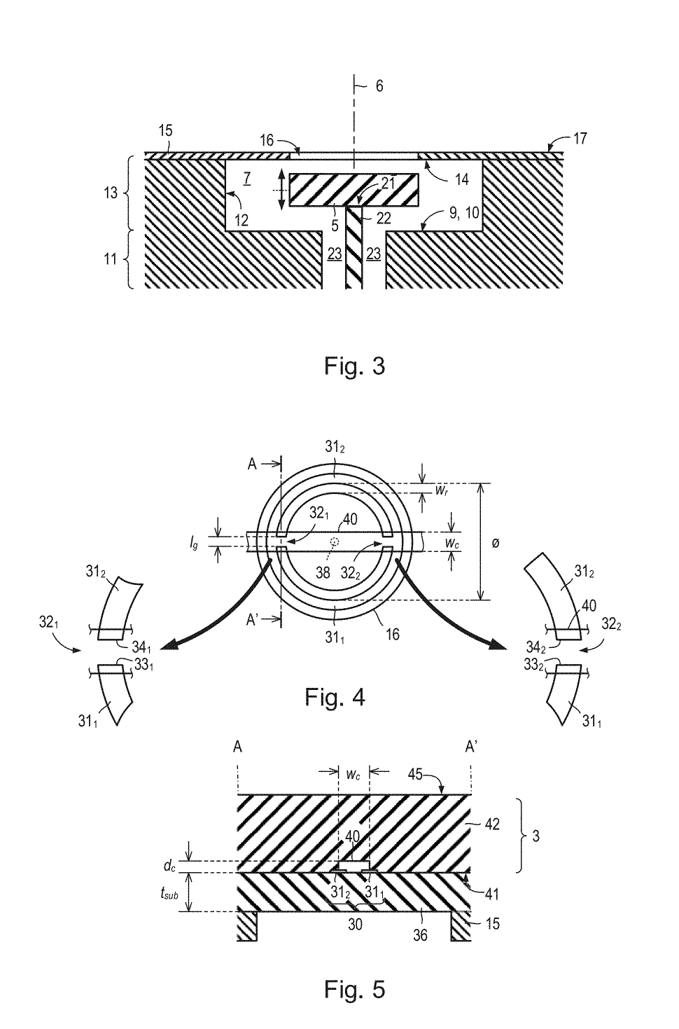

Referring also to FIG. 3, the sensor 2 includes a short, cylindrical (or “disk-shaped”) dielectric resonator 5 having a central axis 6 and disposed in a cylindrical, metal-walled cavity 7 formed by a multi-piece metal housing 8. The dielectric resonator 5 and the cavity 7 are co-axially aligned.

The cavity 7 is defined by a central portion 9 of an upper face 10 of an annular metal base 11, an inner surface 12 of an annular metal wall 13 upstanding from the upper face 10 of the base 11 and a lower face 14 of a removable metal lid 15 having a central aperture 16 (or “through hole”) between an upper face 17 and the lower face 14 of the lid 15. The annular metal base 11 and annular wall 13 are formed in a single piece. The annular wall 13 bounds the central portion 9 of the upper face 10 of the base 11.

The housing 7, i.e. the base 11, wall 13 and lid 15, is formed from copper, although other materials or combination of materials having suitably high electrical conductivity may be used, such as silver-plated aluminium. The base 11 is sandwiched between the lid 15 and a screw holder 18, and is secured by screws (not shown) passing through screw holes 19, 20 in the base 11 and lid 15.

Referring in particular to FIG. 3, the dielectric resonator 5 is attached to a distal end 21 is (shown in FIG. 3 as the top end 21) of a dielectric rod 22 (or “post”) which passes through a central lumen 23 of the annular metal base 10. The dielectric rod 22 is made from a dielectric material, such as quartz, which is different from that of the dielectric resonator 5. The dielectric rod 22 can be controllably raised or lowered (for instance, manually using a screw micrometer or electromechanically using a stepper motor) over a length of travel which is may be between 0.1 and 10 mm and to a precision which may be between 10 and 100 μm. This allows the dielectric resonator 5 to be controllably raised and lowered by a corresponding amount. Thus, the position of the dielectric resonator 5 within the cavity 7 can be varied so as to vary coupling with a split-ring resonator 30.

The dielectric resonator 5 is formed from barium zinc tantalate (BaZnTa) having a relative permittivity, εᵣ, of about 28. However, other suitable dielectric materials, such as CaTiO, BaMgTa, BaZnNbO, AlO or single crystalline dielectric materials like sapphire, can be used. The dielectric resonator 5 has a diameter, d_{DR}, of 8 mm and thickness, t_{DR}, of 4 mm, although these dimensions may be varied. For example, the dielectric resonator 5 may have a diameter, d_{DR}, of 7 mm and thickness, t_{DR}, of 2 mm. The dielectric resonator 5 has an unloaded quality factor, Q, of 7,000 and a resonant frequency, f_{c}, of 9.9 GHz.

Referring in particular to FIG. 1, first and second coupling loops 24, 25 in the form of coaxial cable ends project inwardly into the cavity 7 from opposite sides of the annular metal wall 13 on either side of the dielectric resonator 5. The coupling loops 24, 25 are connected to a measurement system 26. The loops 24, 25 and system 26 are arranged to excite a transverse electric mode in the dielectric resonator 5 having rotational symmetry, i.e. TE_{o,n,p+δ}, where n and p are positive integers, and to measure changes in response.

Referring to FIGS. 1, 2 and 4, the coupled resonator arrangement 2 includes a split-ring resonator 30 comprising first and second metal half rings 311, 312 (or “semi-circular annular sectors”) defining first and second gaps 321, 322. Each gap 321, 322 is formed between a respective pair of facing ring ends 331, 341, 332, 342.

The split ring resonator 30 is supported on a first face 35 of a substrate 36 which in this case takes the form of a glass slide. The substrate 36 may be formed from a plastic, such as polyethylene terephthalate (PET). As will be explained in more detail later, the gaps 321, 322 provide sensing regions. The split-ring resonator 30 is formed from a gold, although other conductive materials, such as copper or silver, may be used. The resonator 30 has an outer diameter, 526 , of 2.45 mm, an annular width, wᵣ, of 0.4 mm and a gap width, w_{g}, of 0.2 mm. The split-ring resonator 30 has a thickness, tₛᵣₒ, of 1 μm and a diameter of 5 mm. The split ring dimensions of the resonator 30 may be varied.

When assembled, the substrate 36 is placed on the upper surface 17 of the lid 15 with the split-ring resonator 30 positioned over the aperture 16 such that the centre 38 of the ring coincides with the central axis 6 of the dielectric resonator 5.

The split-ring resonator 30 is separated from the dielectric resonator 5 by a distance, s, which is at least the thickness, t_{sub}, of the substrate 34. The distance, s, may lie in a range between 0.1 and 1 mm. As will be explained in more detail later, the split-ring resonator 30 and substrate 34 are thin and, thus, have a low profile not only in that that the split-ring resonator 30 can be brought close to the dielectric resonator 5 (e.g. to a separation less than or equal to 10 mm), but also in that the microfluidic chip 3 can be brought very close to the split-ring resonator 30 (e.g. to a separation of less than or equal to 5 mm, or even into direct contact).

Referring to FIGS. 1 and 5, the microfluidic chip 3 comprises at least one groove-like channel 40 formed in a first face 41 of block 42 of plastic material, such as polydimethylsiloxane (PDMS) or polyurethane (PE). The plastic material is preferably biocompatible. The chip 3 may include first and second spaced-apart ports 43, 44 passing from a second, opposite face 45 of the block 42 through to first and second sections or ends of the channel 40. The channel 40 has a width, w_{c}, of about 0.2 mm mm and depth, d_{c}, of about 0.05 mm. The dimensions of the channel 40 may be varied.

Referring to FIGS. 1, 4 and 5, the microfluidic chip 3 is placed face down onto the split-ring resonator 30 and the substrate 36, thereby closing the open top of the channel 40. Furthermore, the channel 40 is arranged such that it runs over the gaps 321, 322. Preferably, the channel 40 is sufficiently wide (i.e. w_{c}>w_{g}) and is suitably positioned such that the channel 40 straddles each gap 321, 322, in other words, at least partially overlaps onto a pair of opposing ends 331, 341 of the half rings 311, 312.

The channel 40 does not need to be arranged in plane with the half rings 311, 312. This can help not only to make the system easier to implement since the microfluidic chip 3 and split-ring resonator do not need to be integrated, but also it can facilitate the alignment of the channel 40 and the split-ring resonator 30.

Referring to FIGS. 1, 2 and 3, the dielectric resonator 5 is arranged inside the metallic microwave cavity 7 or other suitable high-quality factor resonator (i.e. Q>1,000), and the microfluidic chip 3 is positioned in the near-field of the cavity 7, without any need for an electrical contact between the cavity 7 and chip 3. As explained earlier, the microfluidic chip 3 contains a microfluidic channel 40 passing through the gap(s) 321, 322 of an integrated metallic split-ring 30 or another suitable type of resonator. The arrangement leads to a coupled resonant mode between an electromagnetic resonance in the dielectric resonator 5 and an electromagnetic resonance in the split-ring resonator 30, which allows accurate measurement of small changes of the resonant frequency of the split ring resonator 30 by sole excitation and detection of a resonant mode in the dielectric resonator. This enables measurements of the dielectric permittivity of a single cell as it passes through the capacitive gap 321, 322 of the split-ring resonator 3o inside an integrated microfluidic channel by recording the signal amplitude and phase data at only one frequency or the resonant frequency and quality factor derived from several points.

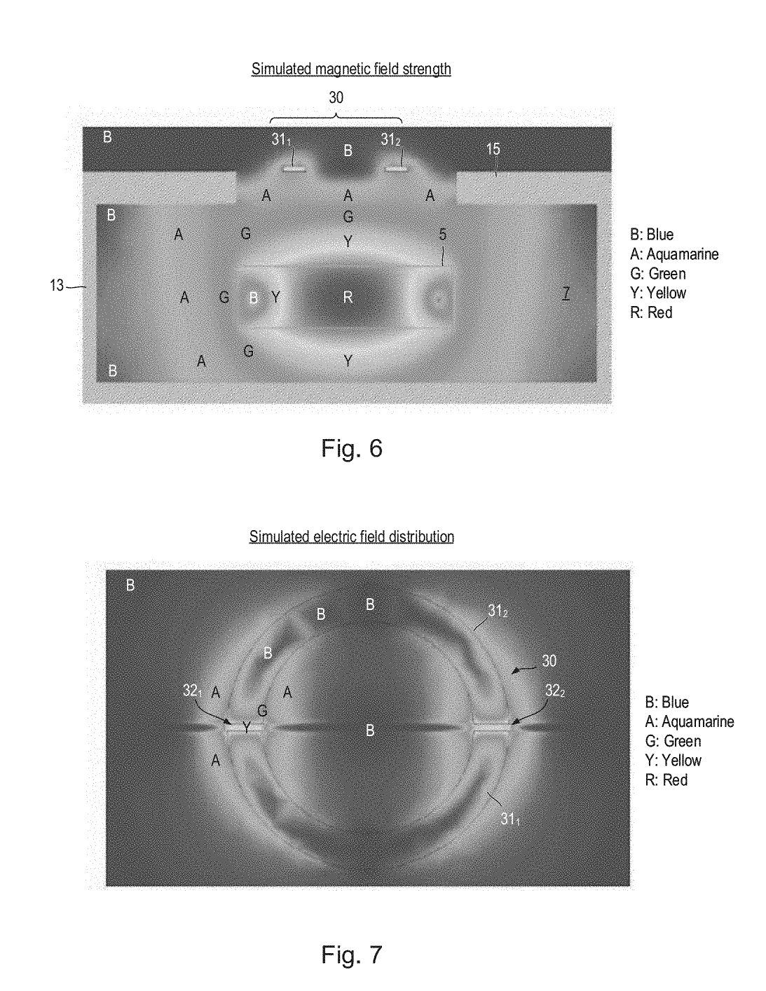

Referring to FIG. 6, a side view of the dielectric resonator 5, cavity 7 and split-ring resonator 30 illustrating simulated magnetic field distribution of a coupled mode is shown illustrating inductive coupling between dielectric resonator and split-ring resonator resonances.

As shown in FIG. 6, there is high field strength which marked with an “R” in the centre of the dielectric resonator 5 which decreases radially, i.e. towards the side wall 13, marked (in order of decreasing field strength) “Y”, “G”, “A” and “B”. The magnetic field strength also decreases longitudinally. However, as shown in FIG. 6, there is reasonable field strength, marked with an “A”, around the half rings 311, 312, which shows inductive coupling.

Referring to FIG. 7, a plan view of the split-ring resonator 30 illustrating simulated electric field distribution of a coupled mode is shown illustrating the electric field enhancement within the capacitive gaps 321, 322 of the split-ring resonator 30.

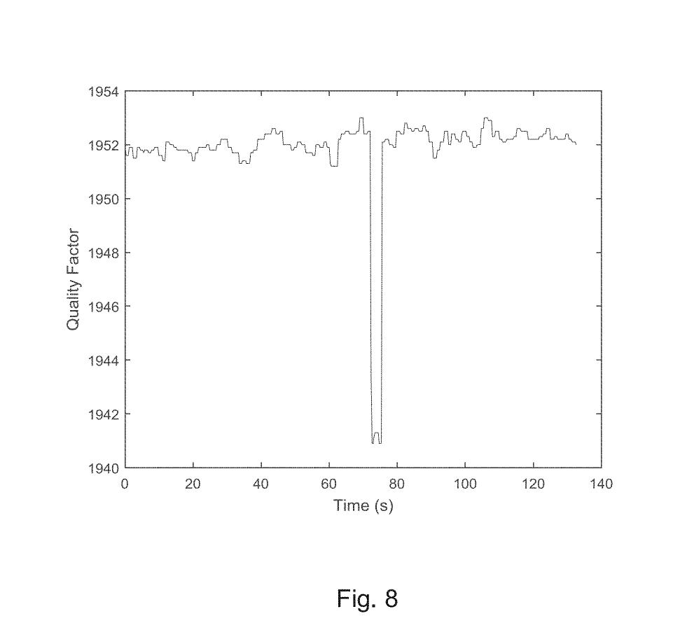

FIG. 8 illustrates experimentally-obtained plot of cavity quality factor, Q, against time showing detection of a 20 μm-diameter polystyrene particle inside a water-filled microfluidic channel as it passes through a capacitive gap 321, 322 (FIG. 2) of the split-ring resonator 30 (FIG. 2).

As shown in FIG. 8, there is a reduction in the value of Q-factor from about 1952 to about 1942 for a period of about 15 s. This drop in Q-factor is attributable to the polystyrene particle passing through the capacitive gap 321, 322 (FIG. 2).

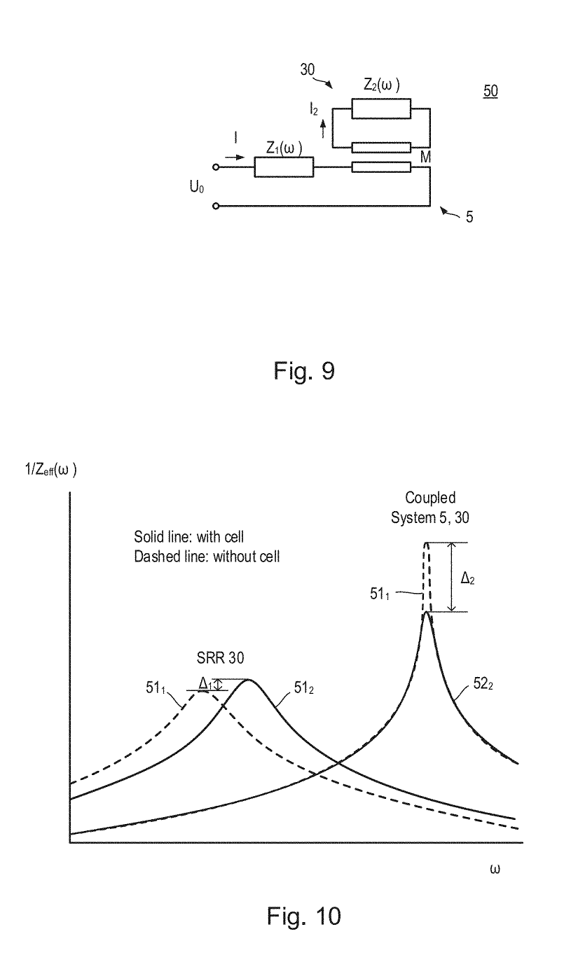

Referring to FIG. 9, an equivalent circuit 50 for the coupled resonator arrangement 2 (FIG. 1) is shown. The dielectric resonator 5 may be modelled using a first frequency-dependent impedance, Z₁(ω), and the split-ring resonator 30 can be modelled using a frequency-dependent impedance, Z₂(ω). The resonators 5, 30 are coupled by mutual inductance, M.

Using this equivalent circuit 50, the behaviour of the coupled resonator arrangement 2 (FIG. 1.) can be modelled.

Referring to FIG. 10, schematic plots 511, 512, 521, 522 of 1/Z_{eff}(ω) for the split-ring resonator 5 and for the coupled resonators 5, 30 are shown.

First and second plots 511, 521 show the responses for the split-ring resonator 5 and for the coupled resonators 5, 30 respectively when no target, e.g. no cell, is present. Third and fourth 512, 522 show the responses for the split-ring resonator 5 and for the coupled resonators 5, 30 respectively when the target is present.

As shown in FIG. 10, there is not only a shift in the resonant peak for the split-ring resonator 30 when the target is present, but also a change in magnitude, Δ₁. There is, however, a much larger change in magnitude, Δ₂, for the coupled resonators 5, 30. This change is much easier to detect. However, without split-ring resonator 5, the dielectric resonator 5 and microfluidic channel 40 (FIG. 1) would require careful alignment. Thus, the split-ring resonator 30 can help to relax the precision of alignment required and can allow sensing of small volumes, for example, 200 μL or less, while the dielectric resonator 5 can provide improved sensitivity.

Referring to FIG. 1, the system 1 can be used for testing samples, for example, to detect the presence of, or count, cancerous cells. The microfluidic chip 3 is a disposable part of the system. For example, each time a new test is conducted, a new microfluidic chip 3 may be used. Optionally, the split-ring resonator 30 may also be disposable part of the system which may be replaced, for example, each time a new test is conducted.

A planar microfluidic chip and thin-film coating and photolithography processes can be used. The width of the microfluidic channel need not be limited as long as the flow of cells passes through the capacitive gap of the split-ring resonator, which can be accomplished by hydrodynamic focussing. Due to the coupling arrangement between the dielectric resonator 5 and split-ring resonator 30, measurements can be performed by recording signal amplitude and phase at just one single frequency, or only several points for resonant frequency and quality factor, which can help to reduce data acquisition time.

The sensor can have one or more advantages. The coupled resonator arrangement combines the advantages of dielectric resonators, namely high quality factor and temperature stable resonances, with the advantages of a planar resonator with integrated capacitors, namely highly sensitive for dielectric measurements on micron-scale objects. As a potential lab-on-chip device, the need to integrate microwave connectors or any electric contacts on the microfluidic chip can be avoided because of the nature of inductive inter-resonator coupling via the magnetic fields of two modes. This feature enables an easy exchange of chips and the realization of low-cost and even disposable chips. A lab-on-chip device with integrated split-ring resonator is arranged outside the cavity and the channels can be observed with a microscope during the measurements. The coupling strength between the dielectric-resonator and the split-ring resonator is mechanically adjustable, which allows easy adjustment of the cavity for different types of split-rings, in order to optimize the system for dielectric measurements of particles of different sizes. The coupling between the dielectric resonator and the split-ring resonances results in a transformation of sample-induced change of the resonant frequency of the split-ring resonator into a change of the quality factor of the dielectric resonator. This can allow the device to operate at a single frequency only (i.e. the resonance frequency of the dielectric resonator) because the resonant frequency of the dielectric resonator is not very sensitive to changes of the effective dielectric permittivity of the sample under test and because the resonant frequency of the dielectric resonator is highly temperature stable. Single frequency measurements enable very fast data acquisition, for example, below 1 millisecond, which allows the device to operate in a flow-cytometer mode.

The sensor herein described may be used in a variety of different applications. It can serve as a microwave flow cytometer for label-free tumour cell detection by assessment of the relative water contend of a cell. Simultaneous cell-size measurements by optical scattering could be combined with the microwave technique in order to separate the cell size—from the cell content contribution to the signal. It can be employed to determine cell content in biological liquids, for example, for pancreatic-or cancer cell detection, in other words, a form of “liquid biopsy”. It can be used for perform concentration measurements on liquids of sub-nanolitre solutions. Blood disorders can be identified, for example, via red and white cell count, and/or platelet count or by detecting rare, unusual red blood cell for early stage infectious disease diagnostics using such a sensor. Realization of low-cost, table-top instruments with integrated microwave electronics and user interface can also be achieved.

It will be appreciated that various modifications may be made to the embodiments hereinbefore described. Such modifications may involve equivalent and other features which are already known in microwave and millimetre-wave resonators and in microfluidics which may be used instead of or in addition to features already described herein. Features of one embodiment may be replaced or supplemented by features of another embodiment.

A dielectric resonator need not be used. For example, other, high-quality factor resonators, such as a cavity resonator, can be used.

Other resonator geometries may be used instead of a split ring resonator, such as a bow tie antenna.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A sensor comprising:
a first resonator operable at microwave and/or millimetre wave frequencies and having a first value of quality factor which is equal to or greater than moo frequency in a range between 1 to 100 GHz; and
a second resonator having a second value of quality factor which is less than the first value of quality factor and which is positioned and orientated with respect to the first resonator so as to be inductively coupled to the first resonator, the second resonator comprising first and second electrically-conductive regions separated by a gap which provides a sensing region.

2. A sensor according to claim 1, wherein the first resonator comprises a dielectric resonator.

3. A sensor according to claim 2, wherein the dielectric resonator comprises a material having a dielectric constant, εᵣ, greater than or equal to 3.

4. A sensor according to claim 1, wherein the first resonator is generally cylindrical.

5. A sensor according to claim 1, wherein the first resonator comprises a conductive housing defining a cavity and an aperture in the housing disposed between the cavity and the second resonator.

6. A sensor according to claim 5, wherein the cavity is generally cylindrical and the aperture is circular.

7. A sensor according to claim 1, wherein the second resonator is generally planar.

8. A sensor according to claim 1, wherein the second resonator comprises a ring comprising at least gap in the ring and is able to support rotational modes around the ring.

9. A sensor according to claim 1, wherein the second resonator is a split-ring resonator.

10. A sensor according to claim 1, wherein the second resonator comprises co-planar thin film regions of conductive material.

11. A sensor system comprising:
a sensor according to claim 1; and
a measurement circuit coupled to the first resonator so as to excite a resonant mode in the first resonator.

12. A sensor system according to claim 11, further comprising:
at least one electrical probe arranged to couple energy into the first and second resonators.

13. A sensor system according to claim 11, further comprising:
at least one magnetic probe arranged to couple energy into the first and second resonators.

14. A sensor system according to claim 11, further comprising:
at least waveguide arranged to couple energy into the first and second resonators.

15. A sensor system according to claim 11, wherein the second resonator comprises a ring comprising at least gap in the ring and is able to support rotational modes around the ring and wherein the measurement circuit is arranged to excite a rotationally-symmetric mode in the second resonator.

16. A sensor system according to claim 11, configured to excite a resonance mode at a frequency which is equal to or greater than 5 GHz, is equal to or greater than 10 GHz, which is equal to or greater than 40 GHz, which is equal to or greater than 100 GHz.

17. A sensor system according to claim 11, configured to excite a resonance mode at a frequency which is equal to or less than 200 GHz or equal to or less than 1 THz.

18. A sensor system according to claim 10, wherein the measurement system is arranged to determine a resonant frequency and a quality factor of a coupled mode using transmission or reflection measurements based on S-parameter analysis of the cavity.

19. A sensor system according to claim 11, further comprising:
a microfluidic chip comprising at least one microfluidic channel, wherein the chip is arranged such that the channel is disposed sufficiently close to sensing region so as to influence a resonant mode in the second resonator.

20. A sensor system according to claim 19, wherein the channel has a side which is exposed directly to the second resonator.
